(19) United States
(12) Patent Application Publication
(10) Pub. No.: US 2004/0191278 A1
(43) Pub. Date: Sep. 30, 2004

(54) Topical agent for application to the skin prior to luminous treatment

(76) Inventor: Flemming Kjaergaard Christensen, Hadsund (DK)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/401,957

(22) Filed: Mar. 31, 2003

(51) Int. Cl. A61K 31/455; A61K 7/00

(52) U.S. Cl. 424/401; 514/356

(57) ABSTRACT

There is disclosed a topical agent in the form of a topical vasodilator for the application to the skin prior to luminous treatment, e.g. laser treatment, wherein said topical vasodilator in a suitable vehicle includes the active substance consisting of nicotinic acid (Niacin) in the range of 0.5-10.0% by weight Preferably, the topical vasodilator is a skin cream containing the active substance, Niacin, in the range of 5% by weight.

### BACKGROUND OF THE INVENTION

1. Field of the Invention

The present invention relates to a topical agent for application to the skin prior to luminous treatment.

2. Description of the Prior Art

Luminous treatment of the skin have been shown to enhance dermal collagen production by way of example by laser treatment by a pulsed dye laser during non-ablative wrinkle treatment by targeting chromophores (haemoglobin) in the microvasculature of the dermis. A mechanism is triggered which ultimately results in an increased production of collagen at the irradiated site.

### SUMMARY OF THE INVENTION

The invention provides a topical agent for application to the skin prior to luminous treatment in order to further enhance the dermal collagen production.

According to the present invention there is provided a topical agent in the form of a topical vasodilator for the application to the skin prior to luminous treatment, which topical vasodilator is in a suitable vehicle including an active substance consisting of nicotinic acid (Niacin) in the range of 0.5-10.0% by weight.

Tests have been carried out for studying the dermal collagen production following topical application of Niacin prior to irradiation with a 585 nm pulsed dye laser. The effect of irradiation by the haemoglobin specific 585 nm pulsed dye laser is significantly increased by the application of topical Niacin at the proposed treatment site prior to treatment. This effect was supported with biochemical analysis which showed an increase in the PIIINP levels above that of irradiation alone. Therefore, a combination of light and topical preparations demonstrate improved efficiency for non-ablative skin rejuvenation. There were no adverse side-effects shown when irradiation was applied to study sites at sub-purpuric levels with fluences at 1.8 J/cm².

The topical vasodilator according to an embodiment of the invention is a skin cream containing 0.5% by weight of the active substance, Niacin.

The topical vasodilator according to another embodiment of the invention is a skin cream containing 2.5% by weight of the active substance, Niacin.

The topical vasodilator according to a further embodiment of the invention is a skin cream containing 5.0% by weight of the active substance, Niacin.

The topical vasodilator according to a still further embodiment of the invention is a skin cream containing 7.5% by weight of the active substance, Niacin.

The topical vasodilator according to a still further embodiment of the invention is a skin cream containing 10.0% by weight of the active substance, Niacin.

In the following the invention is explained in more details by means of a number of detailed examples of skin creams containing the active substance Niacin:

### EXAMPLE 1

A skin cream containing:

| Carpopol ETD 2020 | 84.50% (thickening agent) |
|---|---|
| Niacin | 0.50% (nicotinic acid) |
| Lubragel DV | 14.35% (viscous agent) |
| Diazolodial Urea | 0.25% |
| Methylparaben | 0.25% |
| Cetyl-alcohol | 0.15% |

The pH-value is adjusted between 4 and 6

### EXAMPLE 2

A skin cream containing:

| Carpopol ETD 2020 | 40.00% (thickening agent) |
|---|---|
| Niacin | 7.50% (nicotinic acid) |
| Lubragel DV | 50.00% (viscous agent) |
| Diazolodial Urea | 0.25% |
| Methylparaben | 0.25% |
| Cetyl-alcohol | 0.15% |

The pH-value is adjusted between 4 and 6

As an alternative to the above-mentioned laser treatment by a 585 am pulsed dye laser it should be mentioned that the luminous treatment of the skin may be effected by irradiation treatment of the skin with a flashlight. However, in both cases the application of the topical vasodilator according to the present invention to the skin prior to the luminous treatment have shown to enhance dermal collagen production.

Finally it should be mentioned that the topical vasodilator according to the invention may be applied to the skin in the form of a solution, a lotion, a gel or a cream containing the active substance, Niacin.

1-6. Cancelled.

7. A topical agent for application to the skin comprising:
a light activated topical vasodilator capable of causing vasodilation of the skin after application of the light activated topical vasodilator to the skin upon application of a sufficient amount of light to the skin to cause the vasodilation of the skin including an active agent consisting of nicotinic acid (Niacin) in the range of 0.5 to 10% by weight of the light activated topical vasodilator.

8. A topical agent in accordance with claim 7 wherein:
the nicotinic acid ranges between 0.5% and 7.5% by weight of the light activated topical vasodilator.

9. A topical agent in accordance with claim 7 wherein:
the nicotinic acid ranges between 0.5% and 5% by weight of the light activated topical vasodilator.

10. A topical agent in accordance with claim 7 wherein:
the nicotinic acid ranges between 0.5% and 2.5% by weight of the light activated topical vasodilator.

11. A topical agent in accordance with claim 7 wherein:
the nicotinic acid ranges between 2.5% and 10% by weight of the light activated topical vasodilator.

12. A topical agent in accordance with claim 7 wherein:
the nicotinic acid ranges between 5% and 10% by weight of the light activated topical vasodilator.

13. A topical agent in accordance with claim 7 wherein:
the nicotinic acid ranges between 7.5% and 10% by weight of the light activated topical vasodilator.

14. A method of skin treatment comprising:
applying the topical agent of claim 7 to the skin and exposing the skin to light sufficient to cause the vasodilation of the skin to enhance collagen at the exposed site of the skin.

15. A method of skin treatment comprising:
applying the topical agent of claim 8 to the skin and exposing the skin to light sufficient to cause the vasodilation of the skin to enhance collagen at the exposed site of the skin.

16. A method of skin treatment comprising:
applying the topical agent of claim 9 to the skin and exposing the skin to light sufficient to cause the vasodilation of the skin to enhance collagen at the exposed site of the skin.

17. A method of skin treatment comprising:
applying the topical agent of claim 10 to the skin and exposing the skin to light sufficient to cause the vasodilation of the skin to enhance collagen at the exposed site of the skin.

18. A method of skin treatment comprising:
applying the topical agent of claim 11 to the skin and exposing the skin to light sufficient to cause the vasodilation of the skin to enhance collagen at the exposed site of the skin.

19. A method of skin treatment comprising:
applying the topical agent of claim 12 to the skin and exposing the skin to light sufficient to cause the vasodilation of the skin to enhance collagen at the exposed site of the skin.

20. A method of skin treatment comprising:
applying the topical agent of claim 13 to the skin and exposing the skin to light sufficient to cause the vasodilation of the skin to enhance collagen at the exposed site of the skin.
